(19) United States
(12) Patent Application Publication
(10) Pub. No.: US 2025/0381239 A1
(43) Pub. Date: Dec. 18, 2025

(54) METHOD FOR IMPROVING SLEEP QUALITY OR INCREASING SLEEP DURATION AND METHOD FOR RELIEVING PSYCHOLOGICAL STRESS

(71) Applicant: TOYO SHINYAKU CO., LTD., Fukuoka (JP)

(72) Inventors: Hidetaka FUJII, Saga (JP); Takumi MORIKAWA, Saga (JP); Chikako OMARU, Saga (JP); Hiroshi TOMOZAWA, Saga (JP); Shinichiro TAKASHIMA, Saga (JP); Tomoyasu KAMIYA, Saga (JP)

(73) Assignee: TOYO SHINYAKU CO., LTD., Fukuoka (JP)

(21) Appl. No.: 18/893,925

(22) Filed: Sep. 23, 2024

(30) Foreign Application Priority Data
Jun. 14, 2024 (JP) 2024-096545
Jun. 14, 2024 (JP) 2024-096547

(51) Int. Cl. A61K 36/8998; A61P 25/18; A61P 25/20

(52) U.S. Cl. CPC A61K 36/8998; A61P 25/18; A61P 25/20

(57) ABSTRACT

In modern times, there has been a demand for the development of foods and beverages having sleep improvement effects or psychological stress relief effects that take naturally derived food ingredients as functional ingredients. In the course of researching the functions of young barley leaves, the present inventors discovered that young barley leaves exert an effect of improving sleep quality or an effect of increasing a sleep duration, and thereby completing a composition having a new function using young barley leaves.

### CROSS-REFERENCE TO RELATED APPLICATION

This application claims the priority benefit of Japan application serial no. 2024-096545, filed on Jun. 14, 2024 and Japan application serial no. 2024-096547, filed on Jun. 14, 2024. The entirety of each of the above-mentioned patent applications is hereby incorporated by reference herein and made a part of this specification.

### TECHNICAL FIELD

The present disclosure relates to a composition for improving sleep or relieving stress characterized by containing young barley leaves.

### RELATED ART

In recent years, due to changes in the social environment, stress associated with interpersonal relationships, disruption in daily rhythms, busy daily life, etc., more and more people feel dissatisfied with their sleep quality. Examples of decline in sleep quality include difficulty in falling asleep, frequent mid-sleep awakening (waking up multiple times during the night), lack of perception of deep sleep, and fatigue at awakening. As a countermeasure to such decline in sleep quality, there is a method of using sleeping pills (pharmaceuticals). Sleeping pills include barbiturate sleeping pills, benzodiazepine sleeping pills, etc. However, these sleeping pills cannot be taken casually because they may cause side effects such as headaches and dizziness.

Thus, as a convenient means to improve sleep quality, foods and beverages containing functional ingredients are being developed. For example, theanine is known as a functional ingredient having sleep improvement effects (Patent Literature 1). Theanine is a compound obtained by synthesis or by extraction and refinement from tea. On the other hand, from the viewpoint of safety, some consumers feel uneasy about consuming unfamiliar compounds. Thus, there is a demand for the development of foods and beverages having sleep improvement effects that take naturally derived food ingredients as functional ingredients and are not obtained by synthesizing or refining specific compounds.

Further, the modern society is a stressful society, and every day we live lives under various stresses. Causes of stress (stressors) are not uncommon, such as stress due to information overload on social media and the like, stress due to interpersonal relationships in organizations such as companies and schools, and stress arising from anxiety about health and the future. Stressors may be broadly categorized into four categories: (1) physical stressors such as heat, cold, and noise; (2) chemical stressors such as nutritional deficiency and oxygen deprivation; (3) physiological stressors arising from pain, fever, injuries, etc.; and (4) psychological (synonymous with “mental”) stressors such as anxiety, fear, anger, and tension arising from interpersonal relationships. Specifically, psychological stressors most frequently occur in daily life. If excessive stress is left unaddressed, it may lead to physical ailments and mental disorders such as depression. Thus, dealing with psychological stress is an important issue in modern society.

As means to prevent or relieve stress, there are recreational activities such as hobbies, exercise, and travel, but all of these require considerable time and expense. Thus, as a convenient means to relieve stress, foods and beverages containing functional ingredients are being developed. For example, 5-aminolevulinic acid phosphate (5-ALA) is known as a functional ingredient used in foods and beverages that relieve stress. On the other hand, from the viewpoint of safety, some consumers feel uneasy about consuming unfamiliar compounds. Thus, there is a demand for the development of foods and beverages having stress relief effects that take naturally derived food ingredients as functional ingredients and are not obtained by synthesizing or concentrating specific compounds.

A young barley leaf is a naturally derived food ingredient that is rich in vitamins, minerals, dietary fiber, etc. and is known to have various functionalities such as improving bowel movements and suppressing a rise in neutral fats. However, conventionally, young barley leaves have not been considered to have stress relief effects. For example, Non-Patent Literature 2 describes that, in an evaluation on the effect of a young barley leaf powder on gastric ulcers (an indicator of stress) according to a water immersion restraint test using rats, administration of a young barley leaf powder alone did not exhibit gastric ulcer suppression effect. Generally, since animal testing serves as an indicator for predicting the efficacy of use in humans, based on the results of this test, it is predicted that young barley leaves would not exhibit stress relief effects in humans.

Herein, the test in Non-Patent Literature 2 imposes a physical strain on animals.

However, in recent years, it has been pointed out that stress tests that impose a physical strain on animals cannot correctly evaluate “psychological” stress. Specifically, it has been pointed out that tests such as the water immersion restraint test or forced swim test impose a physical strain on animals, and the test results reflect this influence, so these tests cannot be considered to be tests that correctly evaluate psychological stress only. For example, Non-Patent Literature 3 shows that, comparing mice subjected to physical stress according to the water immersion restraint test with mice subjected only to psychological stress without any physical stimulation on animals, the mechanisms of ulcer formation caused by stress in the two and the effects of drugs on respective ulcers are totally different from each other. This literature describes that famotidine, which was effective against physical stress ulcers, had no effect on psychological stress ulcers, and sulpiride, which was effective against psychological stress ulcers, had no effect on physical stress ulcers. Similarly, Non-Patent Literature 4 describes that animal tests that impose a physical strain, such as the forced swim test, or induce fear through electric shock stimulation cannot correctly evaluate psychological stress due to the influence of pain stimuli accompanying the physical strain or the electric shock stimulation. Further, this literature states that if psychological stress is to be evaluated according to an animal testing, it should be evaluated with stress-related behaviors induced only by psychological stress as indicators.

### RELATED ART LITERATURES

### Patent Literatures

[Patent Literature 1] International Patent Application Publication No. WO01/074352 [Non-Patent Literatures]
[Non-Patent Literature 1] PR TIMES, “Popular 5-ALA as Approach to Depressed Mood! Foods with Function Claims ‘ALA Plus Mental Care’ Newly Released on July 6”, URL: https://prtimes.jp/main/html/rd/p/000000069.000008331.html, Jul. 6, 2021, Retrieved on Apr. 23, 2024
[Non-Patent Literature 2] Akira Nishimura et al., “Preventive Effect of Young Barley Leaf Powder on Stress-Induced Ulcers”, Journal of Japan Mibyou System Association, Vol. 8, No. 2, pp. 207-209, 2002
[Non-Patent Literature 3] Kazuhiko Nomura, “Pharmacological Study on Mechanism of Gastric Mucosal Lesions Induced by Psychological Stress”, Summary of Doctoral Dissertation, The University of Tokyo, Doctor of Veterinary Medicine, Apr. 15, 1996, URL: http://gakui.dl.itc.u-tokyo.ac.jp/cgi-bin/gazo.cgi?no=212829, Retrieved on Apr. 23, 2024
[Non-Patent Literature 4] Shigeo Miyata et al., “Elevated open-platform test: simple method for evaluation of stress vulnerability in mice”, Folia Pharmacologica Japonica, Vol. 132, pp. 213-216, 2008, URL: https://www.jstage.jst.go.jp/article/fpj/132/4/132_4_213/_pdf, Retrieved on Apr. 23, 2024

### SUMMARY OF INVENTION

### Problem to be Solved by Invention

The present disclosure has been made in view of the above-described problems, and the problem to be solved by the present disclosure is to provide a composition including new functions using young barley leaves.

### Means for Solving Problem

In the course of researching the functions of young barley leaves, the inventors of the present disclosure have discovered that young barley leaves exert an effect of improving sleep quality or an effect of increasing a sleep duration, and have thus completed the present disclosure. Further, in the course of researching the functions of young barley leaves, the inventors of the present disclosure have discovered that young barley leaves exert an effect of relieving psychological stress or an effect of reducing fatigue perceived in daily life and the like, and have thus completed the present disclosure.

In other words, the present disclosure is as follows.

[1]

A composition for improving sleep characterized by taking young barley leaves as an active ingredient.

[2]

A composition for improving sleep quality characterized by taking young barley leaves as an active ingredient.

[3]

A composition for reducing sleepiness during the day and at awakening, a composition for improving falling asleep, a composition for suppressing mid-sleep awakening during sleep, reducing sleepiness during the day and at awakening, a composition for reducing fatigue during the day and at awakening, a composition for enhancing satisfaction with sleep, and a composition for improving REM sleep and/or deep sleep, characterized by including young barley leaves as an active ingredient.

[3]

The composition according to any one of [1] to [3], characterized in that young barley leaf-derived dietary fiber is the active ingredient.

[4] The composition according to any one of [1] to [3], characterized in that an intake amount of young barley leaves per day is 0.25 g or more.
[5]

The composition according to any one of [1] to [4], characterized in that an intake amount of young barley leaf-derived dietary fiber per day is 0.125 g or more.

[6]

The composition according to any one of [1] to [5], characterized by being a food with function claims or a food for specified health uses.

[7]

A food or beverage containing a young barley leaf powder, the food or beverage taking young barley leaf-derived dietary fiber as an active ingredient, and the food or beverage indicating one or more functionalities selected from improvement of sleep quality, improvement of falling asleep, enhancement of satisfaction with sleep, improvement of perception of sleep, improvement of perception of deep sleep, improvement of depth of sleep, reduction of the number of times of mid-sleep awakening, reduction of fatigue during the day or at awakening, and improvement of perception of extended sleep duration.

[8] The food or beverage according to [7], characterized in that an intake amount of young barley leaf-derived dietary fiber per day is 0.125 g or more.

The food or beverage according to [7] or [8], characterized by being a food with function

[9] claims or a food for specified health uses.
[10]

A use of young barley leaves for producing a composition for improving sleep.

[11]

A method for improving sleep quality or increasing a sleep duration in a living body of an individual, including a step of having the individual orally ingest young barley leaves.

[12]

A method for improving sleep quality or increasing a sleep duration by causing a human to ingest young barley leaves.

[13]

The method according to [12], achieving, by improving sleep quality or increasing a sleep duration, one or more effects selected from improvement of falling asleep, enhancement of satisfaction with sleep, improvement of perception of sleep, improvement of perception of deep sleep, improvement of depth of sleep, reduction of the number of times of mid-sleep awakening, reduction of sleepiness during the day or at awakening, reduction of fatigue during the day or at awakening, and improvement of perception of extended sleep duration.

Further, the present disclosure is as follows.

[1] A composition for relieving psychological stress characterized by taking young barley leaves as an active ingredient.
[2]

The composition for relieving psychological stress according to [1], characterized in that young barley leaf-derived dietary fiber is the active ingredient.

[3]

The composition for relieving psychological stress according to [1] or [2], characterized in that an intake amount of young barley leaves per day is 0.25 g or more.

[4]

The composition for relieving psychological stress according to any one of [1] to [3], characterized in that an intake amount of young barley leaf-derived dietary fiber per day is 0.125 g or more.

[5]

The composition for relieving psychological stress according to any one of [1] to [4], characterized in that the psychological stress is one or more selected from stress due to office work, stress in daily life, temporary stress, reduction of temporary mood decline, reduction of tension, reduction of irritability, reduction of decrease in vigor and activity, reduction of confusion, fatigue due to office work, fatigue perceived in daily life, and temporary fatigue.

[6] The composition for relieving psychological stress according to any one of [1] to [5], characterized by being a food with function claims or a food for specified health uses.
[7]

A composition for reducing fatigue characterized by taking young barley leaves as an active ingredient.

[8]

A food or beverage containing a young barley leaf powder,
the food or beverage taking young barley leaf-derived dietary fiber as an active ingredient, and
the food or beverage indicating one or more functionalities selected from reduction of psychological stress, reduction of stress due to office work, reduction of stress in daily life, reduction of temporary stress, reduction of fatigue due to office work, reduction of fatigue perceived in daily life, and reduction of temporary fatigue.
   [9]

The composition for relieving psychological stress according to [7], characterized in that an intake amount of young barley leaf-derived dietary fiber per day is 0.125 g or more.

[10]

The composition for relieving stress according to [1] or [2], characterized by being a food with function claims or a food for specified health uses.

[12]

A use of young barley leaves for producing a composition for relieving psychological stress.

[13]

A method for relieving psychological stress in a living body of an individual, including a step of having the individual orally ingest young barley leaves.

[14]

A method for relieving psychological stress by causing a human to ingest young barley leaves.

[15]

The method according to [14], achieving, by relieving psychological stress, one or more effects selected from reduction of psychological stress, reduction of stress due to office work, reduction of stress in daily life, reduction of temporary stress, reduction of temporary mood decline, reduction of tension, reduction of irritability, relief of decline in vigor and activity, reduction of confusion, reduction of fatigue due to office work, reduction of fatigue perceived in daily life, and reduction of temporary fatigue.

### Effects of Invention

The composition of the present disclosure can improve sleep quality or increase a sleep duration. Further, the composition of the present disclosure can relieve psychological stress.

### DESCRIPTION OF EMBODIMENTS

A composition for improving sleep of the present disclosure is characterized by containing young barley leaves. The composition for improving sleep of the present disclosure can improve sleep quality or increase a sleep duration. In the present disclosure, improvement of sleep quality is a concept that includes an effect of improving sleep quality or an effect of preventing or alleviating deterioration of sleep quality.

A composition for relieving psychological stress of the present disclosure is characterized by containing young barley leaves. The composition for relieving psychological stress of the present disclosure can relieve psychological stress by reducing a strain that occurs in the mind and body when a psychological stressor such as tension arising from interpersonal relationships is applied. In the present disclosure, relief of psychological stress is a concept that includes an effect of relieving, alleviating (synonymous with relieving), or preventing psychological stress.

### 1. Young Barley Leaves

The present disclosure is characterized by containing young barley leaves. Thought to originate from Central Asia, Barley (Hordeum vulgare L.) is a poaceous annual or biennial plant and is broadly classified into two-rowed barley, six-rowed barley, etc. based on the shape of its ears. While the seed of barley is often used as a grain, the present disclosure utilizes its young leaves. Comparing the two, the seed of barley is mainly composed of starch (carbohydrates), whereas a young barley leaf is mainly composed of dietary fiber and protein, and the component compositions are totally different from each other. A barley variety used for the young barley leaves of the present disclosure is not particularly limited as long as it is commonly available, and young barley leaves of any variety of two-rowed barley, six-rowed barley, etc. may be used. Young barley leaves refer to leaves of barley before start of ear emergence, and in the present disclosure, stem may be included along with young leaves.

As the young barley leaves, for example, a ground product, a juice, an extract, an extraction residue, etc. may be used. Examples of the ground product may include a dried powder (young barley leaf powder), a finely chopped product, a dried product (dried finely chopped product) thereof, etc. The juice and the extract may be in a liquid form, and a juice powder is preferable. The juice powder of young barley leaves may be obtained by, for example, concentrating juice of young leaves at a low temperature to concentrate the solid content, and freeze-drying or spray-drying the concentrate liquid. For the extract, for example, water (warm water, hot water), ethanol, and aqueous ethanol may be used. The extraction residue may be, for example, a precipitate obtained by suspending a young barley leaf powder in water, stirring and shaking the suspension, and then performing centrifugation. Although the extraction residue is a fraction that is removed in the process of obtaining the juice and the extract, the extraction residue contains abundant young barley leaf-derived dietary fiber and can thus exhibit the effects of the present disclosure.

In the case of using a young barley leaf powder (ground young barley leaf powder) as the young barley leaves of the present disclosure, there is an advantage of being capable of being produced inexpensively. For example, a young barley leaf juice powder is obtained by extracting soluble components such as water-soluble vitamins by juicing and removing insoluble components, and performing drying by evaporating water. However, since insoluble dietary fiber, which is the main component of young barley leaves, is removed, the amount of the young barley leaf juice powder obtained is small compared to the amount of young barley leaves (fresh leaves) which are the raw material. The same also applies to the extract of young barley leaves. In contrast, since the young barley leaf powder (ground young barley leaf powder) is obtained by drying and grinding young barley leaves (fresh leaves) without removing insoluble components, in the case of producing from the same amount of young barley leaves (fresh leaves) as the raw material, a larger amount can be produced compared to the young barley leaf juice powder, and the production cost per unit weight is lower. Furthermore, although less young barley leaves (fresh leaves) are used as the raw material than in the case of the young barley leaf juice powder, the young barley leaf powder (ground young barley leaf powder) exerts effects equal to or greater than the young barley leaf juice powder, as will be illustrated in an animal testing to be described later. Accordingly, as the young barley leaves used in the present disclosure, the ground product is preferable, and among ground products, a dried powder (young barley leaf powder) is particularly preferable, from the viewpoint of being capable of being produced inexpensively without removing insoluble components and being capable of highly exhibiting the effects of the present disclosure by including the young barley leaf-derived dietary fiber.

In the case of using the young barley leaf powder, for example, young barley leaves that are dried to a moisture content of 10 mass % or less, preferably 8 mass % or less, may be used.

The young barley leaf powder used in the present disclosure preferably has a median diameter of 5 μm or more and 50 μm or less, and more preferably 8 μm or more and 30 μm or less. The median diameter refers to a particle size at a cumulative 50% point in a particle size distribution of the powder, and specifically, is a particle size at a cumulative 50% (×50) point in a particle size distribution measured using LMS-300 or LMS-350 manufactured by Seishin Enterprise Co., Ltd., which is a laser diffraction and scattering particle size distribution analyzer. Further, from the viewpoint of being capable of further exhibiting the effects of the present disclosure, the young barley leaves used in the present disclosure preferably include dietary fiber of 10 mass % or more, more preferably 20 mass % or more, and even more preferably 30 mass % or more. An upper limit thereof may be, for example, 80 mass %. The dietary fiber referred to herein is a total content of insoluble dietary fiber and water-soluble dietary fiber. The content of the dietary fiber is measured according to the Prosky method.

Further, from the viewpoint of being capable of further exhibiting the effects of the present disclosure, the young barley leaves used in the present disclosure preferably include insoluble dietary fiber of 5 mass % or more, more preferably 10 mass % or more, and even more preferably 20 mass % or more. An upper limit thereof may be, for example, 70 mass %. The content of insoluble dietary fiber is measured according to the modified Prosky method.

To configure the dietary fiber content and the insoluble dietary fiber content within the above ranges, in the production method of the young barley leaves, the young barley leaf powder may be prepared by grinding young barley leaves without performing juicing and extraction.

### 2. Composition for Improving Sleep

In the present disclosure, sleep improvement refers to improving sleep quality and/or increasing a sleep duration. Improvement of sleep quality refers to shortening a sleep latency (duration from wakefulness to falling asleep), reducing the number of times and a duration of mid-sleep awakening, increasing a proportion of non-rapid eye movement sleep (non-REM sleep) and deep sleep (referring to sleep in stages 3 and 4 in which sleep is particularly deep in the case where non-REM sleep is divided into four stages based on sleep depth; also called slow-wave sleep) in the sleep duration, etc. With the improvement of sleep quality, the following effects can be obtained: reducing sleepiness at awakening and during the day, enhancing satisfaction with sleep and perception of deep sleep, improving perception of extended sleep duration, refreshing awakening, and alleviating fatigue.

On the other hand, increase of the sleep duration refers to increasing a duration (total sleep direction) that is calculated by subtracting a sleep latency (duration from going to bed to falling asleep) and a mid-sleep awakening duration (total of wakefulness duration in the sleep duration) from a duration in bed (duration from going to bed to waking up). For example, in the case where the sleep latency is shortened and the case where the mid-sleep awakening duration is reduced, the sleep quality improves and, at the same time, the total sleep duration also increases. Accordingly, improvement of sleep quality and increase of the sleep duration are closely interrelated concepts.

As shown in a clinical test using the OSA Sleep Inventory to be described later, by ingesting the composition of the present disclosure, factors including “sleepiness at awakening”, “onset and maintenance of sleep”, “dreaming” (indicator for good perception of sleep), “recovery from fatigue”, and “sleep duration” are improved. Accordingly, the composition of the present disclosure may be used as a composition for improving sleep quality, a composition for reducing sleepiness during the day and at awakening, a composition for improving falling asleep, a composition for suppressing mid-sleep awakening during sleep (suppressing the number of times and/or a duration of mid-sleep awakening), a composition for reducing fatigue during the day and at awakening, a composition for enhancing satisfaction with sleep, and a composition for improving REM sleep and/or deep sleep (increasing a duration of REM sleep and/or deep sleep or a proportion of REM sleep and/or deep sleep in a total sleep duration).

The composition for improving sleep of the present disclosure includes young barley leaves, and is not particularly limited as long as it can be distinguished, as a product, from other products in terms of being used for sleep improvement. For example, products that indicate a function of improving sleep on any of the body, packaging, instructions, and promotional materials (advertising media) of the product according to the present disclosure are included in the scope of the present disclosure. The composition for improving sleep of the present disclosure may be products that indicate young barley leaves or components derived from young barley leaves (e.g., young barley leaf-derived dietary fiber) as active ingredients, but is not limited to products that indicate young barley leaves or components derived from young barley leaves (e.g., young barley leaf-derived dietary fiber) as active ingredients on the packaging and the like of the product. For example, it is also possible that the active ingredients are not specified. As the composition for improving sleep with young barley leaf-derived dietary fiber as the active ingredient, it would be sufficient to contain young barley leaf-derived dietary fiber, and it is not necessarily required to use fractionated young barley leaf-derived dietary fiber. For example, a composition for improving sleep with young barley leaf-derived dietary fiber as the active ingredient may be obtained by adding young barley leaves containing young barley leaf-derived dietary fiber, such as a young barley leaf powder (ground powder). Further, even general foods that are produced and sold with a suggested use are included in the scope of the present disclosure. For example, foods sold with testimonials mentioning sleep improvement as personal impressions of those who ingested the food posted on websites and the like are also included in the scope of the present disclosure. Further, foods with function claims that take research papers showing sleep improvement by young barley leaves as scientific evidence for function and indicate young barley leaves or components derived from young barley leaves as functional ingredients are also included in the scope of the present disclosure.

Specifically, in the so-called health foods, examples may include products indicating functions and products indicating being reported to have functions including “enhancing sleep quality”, “contributing to improving sleep quality”, “improving sleep quality”, “supporting good (healthy) sleep”, “contributing to good (healthy) sleep”, “aiding good (healthy) sleep”, “reducing (lessening/improving) (temporary) sleepiness during the day”, “(enhancing/contributing to improving/improving) satisfaction with sleep at awakening”, “improving (reducing) sleepiness at awakening”, “(contributing to) refreshing awakening”, “(contributing to) refreshing and pleasant awakening (at awakening)”, “bringing about deep sleep quickly”, “(contributing to) improving falling asleep”, “(enhancing/contributing to/improving) perception of extended sleep duration”, “reducing mid-sleep awakening duration (during the night)”, “reducing the number of times of mid-sleep awakening (during the night)”, “(contributing to) not waking up during sleep”, “(enhancing/contributing to improving/improving) perception of sleep”, “(enhancing/contributing to improving/improving) perception of deep sleep”, “deepening sleep (further)”, “capable of sleeping soundly”, “(enhancing/contributing to improving/improving) depth of sleep”, “increasing (enhancing) (proportion of) deep sleep”, “increasing (enhancing) (proportion of) REM sleep”, “alleviating (reducing) fatigue at awakening (after awakening)”, etc.

### 3. Composition for Relieving Psychological Stress

In the present application, “stress” refers to a strain that occurs in the mind and body when a stimulus (also called a stressor) that is a cause of stress is applied to a living organism. Stressors may be broadly classified into four categories: (1) physical stressors such as heat, cold, and noise; (2) chemical stressors such as nutritional deficiency and oxygen deprivation; (3) physiological stressors arising from pain, fever, injuries, etc.; and (4) psychological (mental) stressors such as anxiety, fear, anger, and tension arising from interpersonal relationships. In the present application, “psychological stress” refers to stress arising from (4) above, i.e., a strain that occurs in the mind and body when a psychological (mental) stressor such as anxiety, fear, anger, and tension arising from interpersonal relationships is applied to a living organism.

Responses triggered by psychological stress may be classified into three aspects including a psychological aspect, a physical aspect, and a behavioral aspect. Examples of stress responses in the psychological aspect include symptoms such as anger, hostility, confusion, bewilderment, depression (low mood, decreased interest and concern), fatigue, inertia, tension, anxiety, irritability, and decreased vigor and activity. Examples of stress responses in the physical aspect include symptoms such as headaches, stiff shoulders, joint pain, and decreased appetite. Examples of stress responses in the behavioral aspect include increased mistakes at work, increased alcohol consumption, and increased smoking. According to the composition of the present disclosure, these stress responses can be relieved.

The composition for relieving psychological stress of the present disclosure includes young barley leaves, and is not particularly limited as long as it can be distinguished, as a product, from other products in terms of being used for relieving psychological stress. For example, products that indicate a function of relieving psychological stress on any of the body, packaging, instructions, and promotional materials (advertising media) of the product according to the present disclosure are included in the scope of the present disclosure. The composition for relieving psychological stress of the present disclosure may be products that indicate young barley leaves or components derived from young barley leaves (e.g., young barley leaf-derived dietary fiber) as active ingredients, but is not limited to products that indicate young barley leaves or components derived from young barley leaves (e.g., young barley leaf-derived dietary fiber) as active ingredients on the packaging and the like of the product. For example, it is also possible that the active ingredients are not specified. As the composition for relieving psychological stress with young barley leaf-derived dietary fiber as the active ingredient, it would be sufficient to contain young barley leaf-derived dietary fiber, and it is not necessarily required to use fractionated young barley leaf-derived dietary fiber. For example, a composition for relieving psychological stress with young barley leaf-derived dietary fiber as the active ingredient may be obtained by adding young barley leaves containing young barley leaf-derived dietary fiber, such as a young barley leaf powder (ground powder). Further, even general foods that are produced and sold with a suggested use are included in the scope of the present disclosure. For example, foods sold with testimonials mentioning relief of psychological stress as personal impressions of those who ingested the food posted on websites and the like are also included in the scope of the present disclosure. Further, foods with function claims that take research papers showing relief of psychological stress by young barley leaves as scientific evidence for function and indicate young barley leaves or components derived from young barley leaves as functional ingredients are also included in the scope of the present disclosure.

Specifically, in the so-called health foods, examples may include products indicating “relieving (lessening/reducing) psychological (mental) stress”, “reducing (relieving/lessening) temporary psychological (mental) stress”, “relieving (lessening/reducing) temporary psychological (mental) stress accompanying office work”, “relieving (lessening/reducing) psychological (mental) stress accompanying transient tasks”, “relieving (lessening/reducing) temporary psychological (mental) stress due to work or study”, “reducing (relieving/lessening) temporary mood decline”, “reducing (relieving/lessening) temporary stress”, “relieving (lessening/reducing) stress due to office work”, “relieving (lessening/reducing) stress accompanying transient tasks”, “relieving (lessening/reducing) stress due to work or study”, “reducing (relieving/lessening) mood decline”, “relieving (lessening/reducing) work stress due to desk work”, “relieving (lessening/reducing) temporary stress (irritability, tension) accompanying work or study”, “reducing (relieving/lessening) work fatigue after desk work”, “relieving (lessening/reducing) fatigue due to work, study, etc.”, “reducing (relieving/lessening) fatigue for those who feel tired in daily life”, “reducing (relieving/lessening) transient mental fatigue”, “reported to relieve (lessen/reduce) psychological (mental) stress”, “reported to have a function of relieving (lessening/reducing) psychological (mental) stress”, “reported to reduce (relieve/lessen) temporary stress”, “reported to have a function of reducing (relieving/lessening) temporary stress”, “reduction of tension”, “reduction of irritability”, “reduction of decreased vigor and activity”, “reduction of sense of confusion”, etc.

The composition for relieving psychological stress of the present disclosure may be used for the purpose of relieving stress and fatigue such as stress due to office work, stress in daily life, temporary stress, fatigue due to office work, fatigue felt in daily life, and temporary fatigue.

### 4. Dosage Form and the Like of Composition

The composition of the present disclosure may be used as an oral composition. The form of the oral composition is not particularly limited, and examples thereof include foods and beverages, quasi-drugs, pharmaceuticals, etc. However, from the viewpoint of daily intake of young barley leaves to supplement daily nutrition, the composition of the present disclosure is preferably used as foods and beverages. The form of the food and beverage composition is not particularly limited, and examples thereof include foods with function claims, foods for specified health uses, health foods, general foods and beverages, etc. However, from the viewpoint of making it easy for consumers to understand the effects of the present disclosure, foods with function claims and foods for specified health uses are preferable, and foods with function claims are most preferable.

Examples of the form of the composition of the present disclosure may include a powder form, a granule form, a liquid form, a tablet form, a capsule form, etc. The composition of the present disclosure may also be in an obvious food form such as jelly, cookie, candy, gum, etc. The young barley leaves of the present disclosure have a good taste and are thus suitable for intake in a form in which the taste is perceived. Thus, the powder form, the granule form, the liquid form, and the tablet form are preferable, and the powder form, the granule form, and the liquid form are particularly preferable.

A content of young barley leaves in the composition of the present disclosure may be appropriately configured in a range that achieves effects thereof. For example, on a dry mass basis, the composition of the present disclosure may contain 0.01 to 100 mass % of young barley leaves, preferably 0.1 to 100 mass %, and more preferably 1 to 100 mass %.

A content of young barley leaf-derived dietary fiber in the composition of the present disclosure may be appropriately configured in a range that achieves effects thereof. For example, on a dry mass basis, the composition of the present disclosure may contain 0.01 to 80 mass % of young barley leaf-derived dietary fiber, preferably 0.1 to 75 mass %, and more preferably 1 to 70 mass %.

An intake amount of young barley leaves in the present disclosure is not particularly limited. However, from the viewpoint of being capable of further exhibiting the effects of the present disclosure, the intake amount of young barley leaves per day for an adult is preferably 0.25 g or more, more preferably 0.5 g or more, and particularly preferably 1.0 g or more. An upper limit thereof is, for example, 30 g, preferably 20 g, and more preferably 10 g.

Further, an intake amount of young barley leaf-derived dietary fiber in the present disclosure is not particularly limited. However, from the viewpoint of being capable of further exhibiting the effects of the present disclosure, the intake amount of young barley leaf-derived dietary fiber per day for an adult is preferably 0.125 g or more, more preferably 0.25 g or more, and particularly preferably 0.5 g or more. An upper limit thereof is, for example, 15 g, preferably 10 g, and more preferably 5 g.

The composition of the present disclosure may be appropriately designed such that a daily intake amount of young barley leaves or young barley leaf-derived dietary fiber becomes the above intake amount. The composition of the present disclosure may be in a form in which the above intake amount is consumed in one dose, or in a form in which the above intake amount is consumed separately in multiple doses. In other words, for example, as a daily portion, the composition of the present disclosure may be contained in one container or separately in multiple (2 to 4) containers.

The composition of the present disclosure may be added with components other than young barley leaves as required and produced according to conventional methods.

### EXAMPLES

Hereinafter, the present disclosure will be described in more detail based on Examples. However, the present disclosure is not limited to these Examples and may take various forms as long as the problem of the present disclosure can be solved.

Test 1. Evaluation on sleep improvement effect (animal testing)

To evaluate the effect of young barley leaves on sleep, an animal testing of administering young barley leaves to mice and measuring a sleep duration (duration until awakening) was conducted.

### <Tested Substances>

### (Young Barley Leaf Powder)

As the raw material, above-ground parts (leaves and stems) of barley harvested at a height of about 30 cm were used. After washing to remove adhered mud and cutting into pieces of about 5 cm in size, the raw material was blanched only once in hot water at 95° C. for about 100 seconds, and was cooled with cold water. The obtained young barley leaves were dried in a dryer with warm air and was coarsely ground to a size of about 1 mm. Furthermore, it was finely ground using a jet mill grinder to produce a young barley leaf powder (median diameter: about 20 μm; moisture content: 5 mass % or less; young barley leaf-derived dietary fiber: 51 mass %).

(Tested Substances Other than Young Barley Leaf Powder)

The following were used as tested substances other than the young barley leaf powder.

Young barley leaf-derived dietary fiber: A young barley leaf-derived dietary fiber was fractionated from the young barley leaf powder obtained according to the method described above and was used as a tested substance.

Young barley leaf juice powder: A powder obtained by mixing young barley leaf juice with dextrin and drying the mixture was used (young barley leaf-derived dietary fiber: 2 mass %). Barley (grain): A commercially available barley flour was used.

Resistant dextrin: A commercially available resistant dextrin was used.

Cellulose: A commercially available cellulose was used.

Fructooligosaccharide: A commercially available fructooligosaccharide was used.

Ground coffee powder: A commercially available ground coffee bean powder was used.

### <Testing Method>

After acclimating male ICR mice at 4 weeks of age for 5 days, the ICR mice were divided into 9 groups (4 mice per group) such that the body weight values on the day of the test were almost uniform. Each tested substance was dissolved or suspended in pure water to concentrations described in Table 1 to prepare tested substance solutions of 10% (w/v) (20% (w/v) for the young barley leaf juice powder). Each tested substance was prepared to be 1 g/kg (2 g/kg for the young barley leaf juice powder) and forcibly administered orally (10 mL/kg) to mice in each group. Thirty minutes later, sodium pentobarbital (40 mg/kg) was administered intraperitoneally. After loss of postural reflex in the mice, the mice were quickly placed in a supine position on a flat platform, and a duration from the loss of postural reflex to recovery (awakening) was recorded as the sleep duration.

TABLE 1

| | | | | | | Compar- | Compar- | Compar- | Compar- | Compar- |
|---|---|---|---|---|---|---|---|---|---|---|
| | | | Example | Example | Example | ative | ative | ative | ative | ative |
| | | Control | 1 | 2 | 3 | Example 1 | Example 2 | Example 3 | Example 4 | Example 5 |
| Tested | Young barley | | 10 | | | | | | | |
| substance | leaf powder | | | | | | | | | |
| solution | Young barley | | | 10 | | | | | | |
| (%(w/v)) | leaf-derived | | | | | | | | | |
| | dietary fiber | | | | | | | | | |
| | Young barley | | | | 20 | | | | | |
| | leaf juice | | | | | | | | | |
| | powder (juice | | | | | | | | | |
| | component:dextrin = | | | | | | | | | |
| | 1:1) | | | | | | | | | |
| | Barley (grain) | | | | | 10 | | | | |
| | Resistant dextrin | | | | | | 10 | | | |
| | Cellulose | | | | | | | 10 | | |
| | Fructooligosaccharide | | | | | | | | 10 | |
| | Ground coffee powder | | | | | | | | | 10 |
| | Pure water | 100 | 90 | 90 | 80 | 90 | 90 | 90 | 90 | 90 |
| | Total | 100 | 100 | 100 | 100 | 100 | 100 | 100 | 100 | 100 |
| Test | Sleep duration | 694 | 1331 | 1208 | 1105 | 672 | 774 | 653 | 711 | 646 |
| results | (seconds) | | | | | | | | | |
| | (mean) | | | | | | | | | |
| | Relative sleep duration | 100.0 | 191.8 | 174.1 | 159.2 | 96.8 | 111.5 | 94.1 | 102.4 | 93.1 |
| | (control set as 100) | | | | | | | | | |
| | Rate of increase | | 91.8 | 74.1 | 59.2 | −3.2 | 11.5 | −5.9 | 2.4 | −6.9 |
| | (% pt) in sleep | | | | | | | | | |
| | duration with | | | | | | | | | |
| | respect to control | | | | | | | | | |

### <Test Results>

The test results are shown in Table 1. In Example 1 to Example 3 in which young barley leaves were administered, an increase in the sleep duration (duration until awakening) was observed compared to a control. According to this result, it was found that the administration of young barley leaves suppressed mid-sleep awakening and improved sleep quality. In contrast, in Comparative Example 1 to Comparative Example 5, no increase in the sleep duration was observed.

Test 2. Evaluation on psychological stress relief effect (elevated platform test) As described in Related Art, Non-Patent Literature 2 concludes that administration of a young barley leaf powder alone is not believed to show gastric ulcer suppression effects according to a water immersion restraint test using rats. However, the inventors of the present disclosure believed that the testing method described in Non-Patent Literature 2 cannot correctly evaluate the effect of young barley leaves with respect to “psychological” stress because a physical strain is imposed on the rats. To evaluate the effect on psychological stress according to an animal testing, it is recommended to evaluate, as an indicator, stress-related behaviors induced solely by a psychological strain without imposing a physical strain on the animal (refer to Non-Patent Literature 3 and Non-Patent Literature 4 as necessary). Thus, the inventors of the present disclosure decided to select the “elevated platform test” proposed in Non-Patent Literature 4 as a method capable of analyzing animal behavior under psychological stress conditions without imposing a physical strain on the animal, to evaluate the effect of young barley leaves on psychological stress. The elevated platform test is a test that evaluates an intensity of stress responses of animals with a freezing behavior observed under high-altitude stress as an indicator, and serves as an indicator of stress resistance.

### <Tested Substances>

### (Young Barley Leaf Powder)

As the raw material, above-ground parts (leaves and stems) of barley harvested at a height of about 30 cm were used. After washing to remove adhered mud and cutting into pieces of about 5 cm in size, the raw material was blanched only once in hot water at 95° C. for about 100 seconds, and was cooled with cold water. The obtained young barley leaves were dried in a dryer with warm air and was coarsely ground to a size of about 1 mm. Furthermore, it was finely ground using a jet mill grinder to produce a young barley leaf powder (median diameter: about 20 μm; moisture content: 5 mass % or less; young barley leaf-derived dietary fiber: 51 mass %).

(Tested Substances Other than Young Barley Leaf Powder)

The following were used as tested substances other than the young barley leaf powder. Young barley leaf-derived dietary fiber: A young barley leaf-derived dietary fiber was fractionated from the young barley leaf powder obtained according to the method described above and was used as a tested substance.

Young barley leaf juice powder: A powder obtained by mixing young barley leaf juice with dextrin and drying the mixture was used (young barley leaf-derived dietary fiber: 2 mass %).

Barley (grain): A commercially available barley flour was used.

Resistant dextrin: A commercially available resistant dextrin was used.

Cellulose: A commercially available cellulose was used.

Fructooligosaccharide: A commercially available fructooligosaccharide was used.

Ground coffee powder: A commercially available ground coffee bean powder was used.

Coffee is known to have a relieving effect on psychological stress and was thus used as a positive control.

### <Testing Method>

After acclimating male ICR mice at 4 weeks of age for 5 days, the ICR mice were divided into 9 groups (4 mice per group) such that the body weight values on the day of the test were almost uniform. Each tested substance was dissolved or suspended in pure water to concentrations described in Table 2 to prepare tested substance solutions of 10% (w/v) (20% (w/v) for the young barley leaf juice powder). Each tested substance was prepared to be 1 g/kg (2 g/kg for the young barley leaf juice powder) and forcibly administered orally (10 ml/kg) to mice in each group. Thirty minutes after administration, the mice were subjected to high-altitude stress, and a duration of observed freezing behavior was measured. Specifically, a transparent acrylic cylinder (11.4 cm in diameter, 18 cm in height) was turned upside down, and a mouse was placed on a bottom part of the acrylic cylinder as a platform and was video-recorded for 10 minutes. A duration of the freezing behavior (a state in which no movement other than breathing was observed) was measured and accumulated using a stopwatch. In the case where the mouse slipped off the platform during the test, the mouse was promptly returned to the platform, and the measurement was continued.

TABLE 2

| | | | Positive | Example | Example | Example | Comparative | Comparative | Comparative | Comparative |
|---|---|---|---|---|---|---|---|---|---|---|
| | | Control | control | 1 | 2 | 3 | Example 1 | Example 2 | Example 3 | Example 4 |
| Tested | Young barley | | | 10 | | | | | | |
| substance | leaf powder | | | | | | | | | |
| solution | Young barley leaf- | | | | 10 | | | | | |
| (%(w/v)) | derived dietary | | | | | | | | | |
| | fiber | | | | | | | | | |
| | Young barley leaf | | | | | 20 | | | | |
| | juice powder | | | | | | | | | |
| | (juice | | | | | | | | | |
| | component:dextrin = | | | | | | | | | |
| | 1:1) | | | | | | | | | |
| | Barley (grain) | | | | | | 10 | | | |
| | Resistant dextrin | | | | | | | 10 | | |
| | Cellulose | | | | | | | | 10 | |
| | Fructooligosaccharide | | | | | | | | | 10 |
| | Ground coffee powder | | 10 | | | | | | | |
| | Pure water | 100 | 90 | 90 | 90 | 80 | 90 | 90 | 90 | 90 |
| | Total | 100 | 100 | 100 | 100 | 100 | 100 | 100 | 100 | 100 |
| Test | Freezing duration | 213 | 148 | 129 | 135 | 153 | 213 | 214 | 232 | 226 |
| results | (seconds) mean | | | | | | | | | |
| | Relative value | 100.0 | 69.5 | 60.6 | 63.4 | 71.8 | 100.0 | 100.5 | 108.9 | 106.1 |
| | with control as 100 | | | | | | | | | |
| | Rate of decrease | | 30.5 | 39.4 | 36.6 | 28.2 | 0.0 | −0.5 | −8.9 | −6.1 |
| | (% pt) in freeze | | | | | | | | | |
| | duration with | | | | | | | | | |
| | respect to control | | | | | | | | | |

### <Test Results>

The test results are shown in Table 2. In Example 1 to Example 3 in which young barley leaves were administered, a decrease in the freezing duration was observed compared to the control. In particular, Example 1, in which the young barley leaf powder was administered, and Example 2, in which the young barley leaf-derived dietary fiber was administered, exhibited a greater decrease in the freezing duration than the positive control. According to this result, it was found that administration of young barley leaves relieves psychological stress. In contrast, decrease in the freezing duration was not observed in Comparative Example 1 to Comparative

### Example 4

Test 3. Evaluation on sleep improvement effect and psychological stress relief effect (clinical trial)

To evaluate the sleep improvement effect and the psychological stress relief effect in humans, a clinical trial described below was conducted. The sleep improvement effect was evaluated using the OSA Sleep Inventory. The OSA Sleep Inventory is composed of five factors, i.e., Factor 1: sleepiness at awakening, Factor 2: onset and maintenance of sleep, Factor 3: dreaming, Factor 4: recovery from fatigue, and Factor 5: sleep duration. Higher factor scores indicate better perception of sleep.

The psychological stress relief effect was evaluated based on POMS2 (registered trademark). POMS2 (Profile of Mood States 2nd Edition) is a registered trademark of Multi-Health Systems Inc. and is a globally trusted psychological test. POMS2 evaluates psychological states based on factors including “anger-hostility”, “confusion-bewilderment”, “depression-dejection”, “fatigue-inertia”, “tension-anxiety”, “vigor-activity”, “friendliness, and “total mood disturbance”.

The specific testing method is described below.

### <Testing Method>

### 1) Test Foods

A tested food was a powdered beverage obtained by mixing maltose with the young barley leaf powder (produced according to the method described in Test 1). A control food did not include the young barley leaf powder, and was a powdered beverage obtained by adding coloring and flavoring agents to maltose to adjust it to be visually indistinguishable from the tested food. A daily intake amount for both the tested food and the control food was set to 5.6 g, with 2.8 g contained in each plain aluminum bag and distributed to tested subjects to ensure blinding for the tested subjects and involved implementers. Table 3 shows calorie and nutritional content values per recommended daily intake amount (2 bags) of the test foods. The young barley leaf-derived dietary fiber included in the tested food was 0.7 g per recommended daily intake amount (2 bags).

TABLE 3

| | Tested food | Control food |
|---|---|---|
| | (2 bags) | (2 bags) |
| Calorie (kcal) | 21 | 22 |
| Protein (g) | 0.5 | 0.0 |
| Lipid (g) | 0.1 | 0.0 |
| Carbohydrates (g) | 4.7 | 5.4 |
| Saccharides (g) | 4.0 | 5.4 |
| Dietary fiber (g) | 0.7 | 0.0 |
| Salt equivalent (g) | 0.002 | 0.000 |

Calorie Energy conversion factor: protein 4, lipid 9, saccharides 4, dietary fiber 2
Protein Nitrogen-to-protein conversion factor 6.25
Dietary fiber Prosky method

### 2) Tested Subjects

Tested subjects were recruited as paid volunteers and consisted of 40 healthy Japanese men and women (10 males, 30 females) at an age of 20 or more and less than 65 who had some sleep-related concerns and did not meet any of the following exclusion criteria.

Exclusion criteria: (1) Individuals suffering from serious liver diseases, kidney diseases, digestive diseases, heart diseases, respiratory diseases, endocrine diseases, and other metabolic diseases, and individuals being treated for these diseases; (2) Individuals having chronic diseases and are taking medications; (3) Individuals having digestive diseases that affect digestion and absorption, and having a past surgical history of digestive organs; (4) Individuals taking medications, health foods, and supplements that may affect sleep and stress; (5) Individuals who consume alcohol excessively; (6) Individuals who cannot abstain from alcohol the day before the test (the day before recording the sleep questionnaire); (7) Individuals having, or suspected of having, sleep apnea syndrome; (8) Individuals having history of treatment, or having history of present illness, of mental disorders associated with sleep disorders; (9) Individuals who sleep in the same room with family members or others and may be awakened during sleep; (10) Individuals having scheduled business trips (domestic or overseas) or travel (domestic or overseas) during the test; (11) Individuals who cannot stop consuming foods containing live bacteria such as lactic acid bacteria, bifidobacteria, and natto bacteria, foods fortified with oligosaccharides and dietary fiber, health foods considered good for constipation improvement, and foods containing large amounts of sugar alcohols, during the test; (12) Individuals who have been regularly consuming normal foods containing lactic acid bacteria, bifidobacteria, and oligosaccharides for the purpose of relieving constipation; (13) Individuals who have reported allergies to the ingredients of the test foods; (14) Individuals having a past medical history or history of present illness of drug dependence and alcohol dependence; (15) Individuals who are pregnant, breastfeeding, or planning to become pregnant; (16) Individuals with extremely irregular eating habits, and individuals with irregular sleep duration and sleep habits due to irregular shift work and night shifts; (17) Individuals who are participating in or intend to participate in other food tests, tests involving drugs, and tests involving application of cosmetics, medications, etc.; (18) Individuals deemed unsuitable as tested subjects by the principal doctor of the test for any other reason.

### 3) Testing Method

This test was conducted as a randomized, double-blind, placebo-controlled, parallel-group comparative study (allocation ratio: 1:1) with a 4-week intake period.

The allocation was performed according to a block randomization method (block size 4) with gender and age as adjustment factors. Two allocated groups were assigned to a tested food group and a control food group by a controller not directly involved in the test. Furthermore, the controller created and sealed a table (key code) describing the allocation results, and kept the table sealed until the key code was disclosed after determining the subjects to be analyzed, thereby ensuring blinding.

During the test, the subjects were made to consume 2 bags (5.6 g) of the test food (tested food for the tested food group, and control food for the control food group) per day dissolved in an appropriate amount of water or hot water.

During the test, as precautions throughout the test period, the subjects were advised: to maintain the same lifestyle as before the test; to avoid using supplements and health foods; to refrain from consuming foods containing live bacteria such as lactic acid bacteria, bifidobacteria, and natto bacteria, foods fortified with oligosaccharides and dietary fiber, health foods considered good for constipation improvement, and foods containing large amounts of sugar alcohols; and not to consume excessive amounts of alcohol (as a guideline, an amount that would cause mental or physical symptoms resulting from drinking to persist until the day after drinking). In addition, as a precaution for each examination, the subjects were advised to avoid consumption of alcohol on the day before all examinations. The tested subjects were required to obtain permission from the test implementing institution to use any medications, except in cases of emergency.

### 4) Examination Items

### (Evaluation Related to Sleep)

Before intake and 4 weeks after start of intake, an evaluation using the OSA Sleep Inventory (MA version) was conducted as an evaluation related to sleep states. The OSA Sleep Inventory was administered at awakening, and response scale values (Zc scores) for five factors, including “Factor 1: sleepiness at awakening”, “Factor 2: onset and maintenance of sleep”, “Factor 3: dreaming”, “Factor 4: recovery from fatigue”, and “Factor 5: sleep duration”, were calculated based on the user manual. “Dreaming” is an indicator in which a lower score indicates poorer sleep quality, and a higher score indicates sound sleep (good perception of sleep).

### (Evaluation Related to Psychological Stress)

Before intake and 4 weeks after start of intake, an evaluation using the POMS2 Japanese version (abridged version for adults) was conducted as an evaluation related to various mood and emotional states. The POMS2 was administered as an evaluation on “the past week including today”, and standardized scores, i.e., T-scores, converted from the scoring results were calculated for seven scales including “anger-hostility”, “confusion-bewilderment”, “depression-dejection”, “fatigue-inertia”, “tension-anxiety”, “vigor-activity”, and “friendliness”, and “total mood disturbance (TMD) scores”.

### 5) Analyzed Subjects

The number of tested subjects enrolled in this test was 40 (10 males, 30 females). There were no dropouts after randomization, and the test began with 40 subjects. There were no discontinuations or dropouts during the test, and the number of tested subjects who completed the test was 40. After the end of the test, 6 tested subjects (4 in the control food group, 2 in the tested food group), with whom issues were considered to occur in data reliability due to non-compliance with precautions in life and the like, were excluded. 34 subjects (10 males, 24 females) were taken as subjected to be analyzed (16 in the control food group, 18 in the tested food group). The reason for meeting the rejection criteria was that issues were considered to occur in data reliability 5 (4 in the control food group, 2 in the tested food group).

### <Test Results>

### 1) Evaluation Related to Sleep

The results of the OSA Sleep Inventory are shown in Table 4. In all five factors including “sleepiness at awakening”, “onset and maintenance of sleep”, “dreaming”, “recovery from fatigue”, and “sleep duration”, the tested food group exhibited a higher rate of change with respect to before intake compared to the control food group. In the evaluation of the OSA Sleep Inventory, higher factor scores indicate better perception of sleep. According to this result, it was confirmed that intake of young barley leaves improves sleep quality in humans as well.

TABLE 4

| | | Control food | | Tested food | |
|---|---|---|---|---|---|
| | | Before | After 4 weeks | Before | After 4 weeks |
| Factor | | intake | of intake | intake | of intake |
| First factor: | Measured value (mean) | 15.0 | 14.1 | 15.6 | 19.8 |
| sleepiness at | Relative value with respect | 100.0 | 94.2 | 100.0 | 126.8 |
| awakening | to before intake | | | | |
| | Rate of change (%) with | | −5.8 | | 26.8 |
| | respect to before intake | | | | |
| Second factor: | Measured value (mean) | 18.2 | 16.7 | 13.8 | 18.1 |
| onset and | Relative value with respect | 100.0 | 91.5 | 100.0 | 131.1 |
| maintenance | to before intake | | | | |
| of sleep | Rate of change (%) with | | −8.5 | | 31.1 |
| | respect to before intake | | | | |
| Third factor: | Measured value (mean) | 25.2 | 23.7 | 23.8 | 24.9 |
| dreaming | Relative value with respect | 100.0 | 94.2 | 100.0 | 104.6 |
| | to before intake | | | | |
| | Rate of change (%) with | | −5.8 | | 4.6 |
| | respect to before intake | | | | |
| Fourth factor: | Measured value (mean) | 16.2 | 17.7 | 15.1 | 20.6 |
| recovery from | Relative value with respect | 100.0 | 109.4 | 100.0 | 136.5 |
| fatigue | to before intake | | | | |
| | Rate of change (%) with | | 9.4 | | 36.5 |
| | respect to before intake | | | | |
| Fifth factor: | Measured value (mean) | 18.2 | 17.9 | 17.5 | 20.4 |
| sleep duration | Relative value with respect | 100.0 | 98.5 | 100.0 | 116.7 |
| | to before intake | | | | |
| | Rate of change (%) with | | −1.5 | | 16.7 |
| | respect to before intake | | | | |

Relative value with respect to before intake: Relative value in the case where the measured value (mean) before intake is set to 100

Analyzed subjects: 16 in the control food group. 18 in the tested food group

### <Test Results>

### 2) Evaluation Related to Psychological Stress

The analysis results of POMS2 are shown in Table 5. Among the evaluation items, higher values for two items “vigor-activity” and “friendliness” and lower values for six items “anger-hostility”, “confusion-bewilderment”, “depression-dejection”, “fatigue-inertia”, “tension-anxiety”, and “TMD scores” indicate a preferable state (a state with less psychological stress). Comparing the rate of change after 4 weeks of intake with respect to before intake, the tested food group exhibited higher values in the two items “vigor-activity” and “friendliness” and lower values in the six items “anger-hostility”, “confusion-bewilderment”, “depression-dejection”, “fatigue-inertia”, “tension-anxiety”, and “TMD scores” compared to the control food group. According to this result, it was confirmed that the effect of relieving psychological stress by intake of young barley leaves is also recognized in humans.

TABLE 5

| | | | Control food | | Tested food | |
|---|---|---|---|---|---|---|
| | | | | After 4 | | After 4 |
| | | | Before | weeks of | Before | weeks of |
| | Item | | intake | intake | intake | intake |
| Item in | Vigor- | Measured value (mean) | 48.8 | 48.9 | 51.2 | 53.6 |
| which | activity | Relative value with respect to before intake | 100.0 | 100.4 | 100.0 | 104.6 |
| high | | Rate of change (%) with respect to before intake | | 0.4 | | 4.6 |
| values | Friendly | Measured value (mean) | 53.2 | 52.9 | 55.4 | 55.7 |
| are good | | Relative value with respect to before intake | 100.0 | 99.4 | 100.0 | 100.5 |
| | | Rate of change (%) with respect to before intake | | −0.6 | | 0.5 |
| Item in | Anger- | Measured value (mean) | 50.8 | 48.0 | 46.6 | 43.9 |
| which | hostility | Relative value with respect to before intake | 100.0 | 94.6 | 100.0 | 94.4 |
| low | | Rate of change (%) with respect to before intake | | 5.4 | | −5.6 |
| values | Confusion- | Measured value (mean) | 51.3 | 51.7 | 51.0 | 47.7 |
| are good | bewilderment | Relative value with respect to before intake | 100.0 | 100.7 | 100.0 | 93.6 |
| | | Rate of change (%) with respect to before intake | | 0.7 | | −6.4 |
| | Depression- | Measured value (mean) | 48.9 | 49.1 | 50.1 | 46.5 |
| | dejection | Relative value with respect to before intake | 100.0 | 100.3 | 100.0 | 92.9 |
| | | Rate of change (%) with respect to before intake | | 0.3 | | −7.1 |
| | Fatigue- | Measured value (mean) | 54.8 | 52.3 | 51.8 | 45.2 |
| | inertia | Relative value with respect to before intake | 100.0 | 95.5 | 100.0 | 87.1 |
| | | Rate of change (%) with respect to before intake | | −4.5 | | −12.9 |
| | Tension- | Measured value (mean) | 51.8 | 50.6 | 51.6 | 47.4 |
| | anxiety | Relative value with respect to before intake | 100.0 | 97.8 | 100.0 | 91.8 |
| | | Rate of change (%) with respect to before intake | | −2.2 | | −8.2 |
| | TMD scores | Measured value (mean) | 52.1 | 50.9 | 49.9 | 45.2 |
| | | Relative value with respect to before intake | 100.0 | 97.7 | 100.0 | 90.6 |
| | | Rate of change (%) with respect to before intake | | −2.3 | | −9.4 |

Relative value with respect to before intake: Relative value in the case where the measured value (mean) before intake is set to 100

### INDUSTRIAL APPLICABILITY

The composition of the present disclosure can exert sleep improvement effects by containing young barley leaves. Since the composition of the present disclosure can be used as foods and beverages for the purpose of improving sleep, it has high industrial applicability. Further. the composition of the present disclosure can exert effects of relieving psychological stress by containing young barley leaves. Since the composition of the present disclosure can be used as foods and beverages for the purpose of relieving psychological stress, it has high industrial applicability.

What is claimed is:

1. A method for improving sleep quality or increasing a sleep duration by causing a human to ingest young barley leaves.

2. A method for relieving psychological stress by causing a human to ingest young barley leaves.
